(19) United States
(12) Patent Application Publication
(10) Pub. No.: US 2012/0125325 A1
(43) Pub. Date: May 24, 2012

(54) THE USE OF AMLEXANOX IN THE THERAPY OF NEUTROPHIL-DRIVEN DISEASES

(76) Inventors: Robin Mark Bannister, London (GB); John Brew, London (GB)

(21) Appl. No.: 13/375,528

(22) PCT Filed: May 28, 2010
(86) PCT No.: PCT/GB10/50905

§ 371 Date: Feb. 6, 2012

(30) Foreign Application Priority Data
Jun. 1, 2009 (GB) 0909352.7
Oct. 26, 2009 (GB) 0918727.9
Oct. 26, 2009 (GB) 0918728.7

(51) Int. Cl. A61M 11/00; A61K 9/12; A61K 31/436; A61P 1/00; A61M 15/00; A61P 9/00; A61P 11/00; A61P 11/08; A61P 11/06; A61P 29/00; A61K 9/00; A61P 17/06

(52) U.S. Cl. 128/200.14; 424/400; 424/45; 514/291; 128/203.12; 128/203.15

(57) ABSTRACT

An agent, which is amlexanox, is useful in the therapy of a disease associated with neutrophilia.

### FIELD OF THE INVENTION

This invention relates to the use of amlexanox in therapy of diseases associated with neutrophilia

### BACKGROUND OF THE INVENTION

Chronic respiratory diseases, including sarcoidosis, chronic obstructive pulmonary disease (COPD), cystic fibrosis (CF), acute respiratory distress syndrome (ARDS) and asthma, constitute a major health problem, but they are poorly treated by current therapies. Such therapies include inhaled corticosteroids, but their use is not always efficacious and may give rise to undesirable side-effects, including systemic side-effects. Many respiratory conditions involve a lung injury component and there are no single agent therapies that are able to treat such diseases. Often, such diseases require two or three co-administered medicines.

Amlexanox is a compound that has been approved for the treatment of mouth ulcers and, as a nasal spray, as an anti-allergy agent. It is disclosed in U.S. Pat. No. 4,143,042; the suggested therapies are of allergic asthma, allergic dermatitis, hay fever and other allergic diseases, and the proposed routes of administration are oral and by injectable solutions, inhalation and ointments.

Respiratory inflammation characterised by eosinophil infiltration, namely asthma, is characterised by reversible loss of lung function with no tissue damage. Asthma is often characterised by increased collagen lay down in lung connective tissue, and does not involve neutrophil infiltration.

Irreversible obstructive lung diseases such as COPD, bronchiectasis and ARDS are strongly associated with destructive lung inflammation. They are characterised by environmental inflammatory triggers such as smoking and infection, resulting in leukocyte infiltration and the release of cytokines, chemokines and a multitude of inflammatory mediators. These mediators cause leukocytes, primarily neutrophils, to release destructive agents such as superoxide anions, matrix metalloproteases and cathepsin E. These neutrophil-derived molecules cause destruction of the lung's gaseous exchange cellular layers and its supporting connective tissue, resulting in progressive and irreversible lung damage and irreversible loss of lung function.

Unlike irreversible obstructive lung diseases, reversible obstructive lung diseases, such as asthma, are mainly characterised by respiratory inflammation characterised by primarily eosinophilic infiltration and reversible loss of lung function with no tissue destruction. Asthma is characterised by bronchial hypersensitivity to triggers (such as cold, exercise and allergens) that causes obstructive bronchospasm. Such diseases are entirely reversible once the trigger is removed or the patient is treated with bronchodilators. Reversible and irreversible obstructive lung diseases are pathologically very different. They involve different parts of the immune system. Reversible obstructive lung disease is driven by activation of the Th2 immune system, while irreversible obstructive lung disease characterised by the activation of the innate immune system. Therefore, irreversible and reversible obstructive lung diseases are thought to require very different therapeutic approaches. For example, inhaled corticosteroids are known to be very effective treatments for the majority of reversible obstructive lung disease patients, while they have little therapeutic effect in irreversible obstructive lung disease.

WO2009/007673 discloses a combination of mast cell inhibitor and a PPARγ agonist, for treating inflammatory disorders. COPD is listed as one of a number of inflammatory disorders and amlexanox is listed as one of a number of mast cell inhibitors. Mast cell inhibitors do not generally inhibit neutrophilia. Oral administration is listed as one of a number of possible routes of administration.

Taniguchi et al, 1990, and Kimishoto et al, 2006, for example, report that amlexanox has effects on neutrophils, which are opposite to the effects that would be beneficial in the treatment of COPD. This is based primarily around its effects on leukotriene B4 and S100A12. Specifically, amlexanox has been shown to increase LTB4 production, which would be detrimental to COPD.

Neutrophils are normally found in the blood stream. However, during the acute phase of inflammation, particularly as a result of bacterial infection and some cancers, neutrophils migrate toward the site of inflammation, firstly through the blood vessels, then through interstitial tissue, following chemical signals (such as Interleukin-8 (IL-8), Interferon-gamma (IFN-gamma), and C5a) in a process called chemotaxis.

Taniguchi et al 1990 reports that amlexanox has effects on neutrophil biology. However, the concentrations of amlexanox used in the vitro experiments are higher than anything that is achievable in vivo.

### SUMMARY OF THE INVENTION

Given the reports in the literature, it was therefore surprising to find that amlexanox, when administered orally, is effective in the therapy of LPS-induced pulmonary neutrophilia. Amlexanox has a history of use in atopic disease such as allergic rhinitis and asthma (Th2 disease). Consequently, it is highly unexpected for such a molecule to inhibit neutrophilia (Th1/innate type inflammation). That is the principle on which the invention is based.

The invention may be of particular value for administration to patients having a chronic respiratory disease, e.g. associated with evidence of infection or inflammation. An advantage of the invention may lie in reduced systemic side-effects associated with the active agent. It has been found that amlexanox has little or no effect as a bronchodilator; therefore, it would not be useful to treat allergic asthma.

Given the prior art, it is surprising that inhaled amlexanox has utility in the therapy of conditions involving destructive lung inflammation, e.g. COPD and ARDS. Both are characterised by inflammatory triggers that result in leukocyte infiltration, the release of cytokines, chemokines and a multitude of inflammatory mediators. These mediators cause leuokocytes, primarily neutrophils, to release destructive agents such as superoxide anions, matrix metalloproteases and cathepsin E. These neutrophil-derived molecules cause destruction of the lung's gaseous exchange cellular layers and its supporting connective tissue, resulting in progressive and irreversible lung damage and irreversible loss of lung function. Unlike asthma, COPD is characterised by activation of the innate immune system.

According to a first aspect, the present invention is therefore amlexanox as an active agent to be delivered orally, for the treatment of a condition associated with neutrophilia.

Amlexanox may also be useful for the treatment of asthma. However, as amlexanox is not a bronchodilator, the subject to be treated should also be receiving treatment with a bronchodilator.

According to a second aspect, the present invention is therefore amlexanox as an active agent to be delivered via the inhaled route, for the treatment of a respiratory disease, provided that the subject of administration is also receiving a bronchodilator if bronchodilation is required for treatment.

### DESCRIPTION OF THE DRAWINGS

FIG. 1 is a bar graph representing the total number of cells in bronchoalveolar lavage fluid of LPS-exposed mice pre-treated either with vehicle, amlexanox or dexamethasone. Amlexanox is administered orally.

FIG. 2 is a bar graph representing the total number of cells in bronchoalveolar lavage fluid of LPS-exposed mice pre-treated either with vehicle, amlexanox or fluticasone. Amlexanox is administered via the inhaled route.

FIG. 3 is a bar graph representing the total number of cells in bronchoalveolar lavage fluid of LPS-exposed mice pretreated either with vehicle, amlexanox, fluticasone, salbutamol or dexamethasone. Each column represents the mean and each bar represents s.e.mean of n=8. Changes were compared to the vehicle control (*) animals using ANOVA followed by Dunnet's test. *P<0.05 and **P<0.01. Combination treatments with amlexanox and salbutamol (#) were compared to amlexanox alone for significance using unpaired t-test. #P<0.05.

FIG. 4 is of bar graphs representing A) the total number of neutrophils in bronchoalveolar lavage fluid of LPS-exposed mice pretreated either with vehicle, amlexanox, salbutamol or RV1088 and B) the percentage contribution of neutrophils of the total cell count in bronchoalveolar lavage fluid of LPS exposed mice pretreated either with vehicle, amlexanox, fluticasone, salbutamol or dexamethasone. Each column represents the mean and each bar represents s.e.mean of n=8. Changes were compared to the vehicle control (*) animals using ANOVA followed by Dunnets test. *P<0.05 and **P<0.01. Combination treatments with amlexanox and salbutamol (#) were compared to amlexanox alone for significance using unpaired t-test. #P<0.05.

FIG. 5 is a scatter graph showing the total number of cells in bronchoalveolar lavage fluid of LPS-exposed mice pretreated either with vehicle, amlexanox, fluticasone, salbutamol or dexamethasone. Each symbol represents total cells for each individual animal per group. n=8 per group.

FIG. 6 is a scatter graph showing the total number of neutrophils in bronchoalveolar lavage fluid of LPS-exposed mice pretreated either with vehicle, amlexanox, fluticasone, salbutamol or dexamethasone. Each symbol represents total neutrophils for each individual animal per group. n=8 per group.

### DESCRIPTION OF THE FURTHER EMBODIMENTS

Any suitable form of the active agent can be chosen. These include salts, prodrugs and active metabolites.

As indicated above, the invention has utility in therapy. An aspect is the manufacture of a medicament for therapy (whether prophylactic or for treatment) of a condition involving destructive lung inflammation, a chronic respiratory disease or an irreversible obstructive lung disease. Conditions that may be treated include chronic obstructive pulmonary disease (COPD), acute respiratory distress syndrome (ARDS), bronchiectasis, chronic bronchitis, pulmonary emphysema or small airway disease. Further conditions that may be treated include sarcoidosis, CF, asbestosis, farmer's lung and silicosis. Asthma may be treated by the use of amlexanox in combination with a bronchodilator. Another aspect is the manufacture of a medicament for the therapy of a systemic disease associated with neutrophilia.

Conditions associated with neutrophilia, which can be treated with amlexanox, are acute infections (bacterial, fungal, spirochete, parasitic, rickettsial and viral infections), collagen diseases (chronic rheumatoid arthritis, Wegener's granulomatosis and Behcet's disease), gout, Gaucher's disease, Cushing's syndrome, myelofibrosis, neoplastic neutrophilia, polycythemia vera, psoriasis, inflammatory bowel disease, ischemia reperfusion injury, thrombosis and glomerulonephritis. The ischemia reperfusion injury may be cardiac in origin (such as myocardial infarction), cerebral in origin (such as stroke) or following an organ transplant (such as following a kidney transplant).

In a particularly preferred embodiment, the condition to be treated is gout.

Therapy according to the invention may be conducted in generally known manner, depending on various factors, such as the sex, age or condition of the patient, and the existence or otherwise of one or more concomitant therapies. The patient population may be important, for example, in the treatment of patients with liver disease.

Administration may be via the oral or inhaled route. In one embodiment, amlexanox is formulated into an oral mucosal paste.

The amount of active agent in one unit dose may be from 10 mg to 1 g. Preferably, the unit dose is from 10 mg to 100 mg. More preferably, the unit dose is from 40 mg to 60 mg.

The agent for the invention may be dosed daily or weekly. If it is dosed daily, that dose may be given as a single dose or as divided doses (with dosing no more frequent than 4 times per day, preferably 2 or 3 times per day).

Preferred dose ranges for daily administration (for both oral and inhaled administration) are from 1 to 500 μg or from 5 to 100 μg of active agent per day.

Preferred unit dose ranges for weekly administration (for both oral and inhaled administration) are form 5 to 3000 μg or from 25 to 500 μg of active agent per week.

For the treatment of conditions such as COPD and CF, it is preferred that the active agent should reach the deep lung. For this purpose, it is preferred that the agent is delivered via the inhaled route in the form of particles up to 10 μm in size, e.g. 0.5 to 10 μm in mass median aerodynamic diameter.

Administration by inhalation is a preferred embodiment. Devices and formulations suitable for delivery by inhalation typically comprise particles of the active agent, and are generally known to the skilled person. In one embodiment, the composition may be prepared for delivery as an aerosol in a liquid propellant, for example for use in a pressurised or other metered dose inhaler (MDI). Propellants suitable for use in a PMDI are known to the skilled person, and include CFC-12, HFA-134a, HFA-227, HCFC-22 (difluorochloromethane), HFA-152 (difluoroethane and isobutane). Nebulisers and aerosol delivery systems are further alternatives.

In another embodiment, a composition of the invention is in dry powder form, for delivery using a dry powder inhaler (DPI). Dry powder inhalers are known. A dry powder for use in the inhalers will usually have a mass median aerodynamic diameter of less than 30 μm, preferably less than 20 μm and more preferably less than 10 μm. Microparticles having aerodynamic diameters in the range of 5 to 0.5 μm will generally be deposited in the respiratory bronchioles, whereas smaller particles, having aerodynamic diameters in the range of 2 to 0.05 μm, are likely to be deposited in the alveoli.

The DPI may be a passive dry powder inhaler, which relies on the patient's inspiration to introduce the particles into the lungs. Active inhalers, requiring a mechanism for delivering the powder to the patient, may also be used.

It will be appreciated that the particulate compositions are to be formulated in physiologically effective amounts. That is, when delivered in a unit dosage form, there should be a sufficient amount of the active agent to achieve the desired response. As the particles are intended primarily for delivery in dry powder inhalers, it will be appreciated that a unit dose comprises a predefined amount of particles delivered to a patient in one inspiratory effort. For guidance only, a single unit dose will be approximately 1 mg to 15 mg, preferably 5 mg to 10 mg of the particles.

The frequency of dosing can be selected by one of ordinary skill in the art. It may be, for example, once or twice daily, or continuous.

The microparticles may also be formulated with additional excipients to aid delivery and release. For example, in the context of dry powder formulations, the microparticles may be formulated with additional large carrier particles which aid the flow from the dry powder inhaler into the lung. Large carrier particles are known, and include lactose particles having a mass median aerodynamic diameter of greater than 90 μm. Alternatively, or in addition, the microparticles may be dispersed within a carrier material. For example, the microparticles may be dispersed within a polysaccharide matrix, with the overall composition formulated as microparticles for direct delivery to the lung. The polysaccharide acts as a further barrier to the immediate release of the active component. This may further aid the controlled release process. Suitable carrier materials will be apparent to the skilled person and include any pharmaceutically acceptable insoluble or soluble material, including polysaccharides. An example of a suitable polysaccharide is xantham gum.

The compositions may also comprise additional therapeutic agents, either as separate components, i.e. as separate microparticles, or combined with the active agent in the microparticles.

Compositions for use in the invention may be produced using conventional formulation techniques. In particular, spray-drying may be used to produce microparticles comprising the active agent dispersed or suspended within a material that provides the controlled release properties.

The process of milling, for example jet-milling, may also be used to formulate a therapeutic composition suitable for use in the invention. The manufacture of fine particles by milling can be achieved using conventional techniques. The term “milling” is used herein to refer to any mechanical process which applies sufficient force to the particles of active material to break or grind the particles down into fine particles. Various milling devices and conditions are suitable for use in the production of the compositions of the invention. The selection of appropriate milling conditions, for example, intensity of milling and duration, to provide the required degree of force, will be within the ability of the skilled person. Ball milling is a preferred method. Alternatively, a high pressure homogeniser may be used, in which a fluid containing the particles is forced through a valve at high pressure, producing conditions of high shear and turbulence. Shear forces on the particles, impacts between the particles and machine surfaces or other particles, and cavitation due to acceleration of the fluid, may all contribute to the fracture of the particles. Suitable homogenisers include the EmulsiFlex high pressure homogeniser, the Niro Soavi high pressure homogeniser and the Microfluidics Microfluidiser. The milling process can be used to provide the microparticles with mass median aerodynamic diameters as specified above. If hygroscopic, the active agent may be milled with a hydrophobic material, as stated above.

If required, the microparticles produced by the milling step can then be formulated with an additional excipient. This may be achieved by a spray-drying process, e.g. co-spray-drying. In this embodiment, the particles are suspended in a solvent and co-spray-dried with a solution or suspension of the additional excipient. Preferred additional excipients include polysaccharides. Additional pharmaceutically effective excipients may also be used.

Therapy according to the invention may be conducted in generally known manner, depending on various factors, such as the sex, age or condition of the patient, and the existence or otherwise of one or more concomitant therapies. The patient population may be important, for example, in the treatment of patients with liver disease. Again, by way of example only, in the treatment of sarcoidosis, the patient may be symptomatic or asymptomatic, and may exhibit other conditions, e.g. acute, chronic and/or life-threatening.

The formulation that is used, if necessary with a bronchodilator, desirably has a bronchodilatory effect over a prolonged period and raises FEV levels. Following initial dosing, and subsequent doses, the FEV₁ level may be maintained at a level higher than that prior to the start of the therapy. The amount of active agent released over this period can be sufficient to provide effective relief (bronchodilation) of the respiratory disease, over a desired period.

The degree of bronchodilation may be determined by techniques known to the skilled person, including spirometry. This may be used to measure the FEV₁ over the administration period. It is desirable to achieve a FEV₁ value that is greater than 10% of the predicted normal value, preferably greater than 20% and most preferably greater than 30%, over the administration period.

The amount of active ingredient in one unit dose may be, e.g. 0.02-5 mg, preferably less than 2 mg, most preferably less than or about 1 mg. Larger or smaller doses may also be provided, for example, less than 100 μg. In the particles, the active agent may be present in, for example, greater than 20% by weight, preferably greater than 40% by weight, and more preferably greater than 60% by weight.

The compounds of the invention are preferably to be administered orally, for example as tables, troches, lozenges, aqueous or oral suspensions, dispersible powders or granules. Preferred pharmaceutical compositions of the invention are tablets and capsules. Liquid dispersions for oral administration may be syrups, emulsions and suspensions. More preferably, the pharmaceutical composition is a pressed tablet or capsule with conventional excipients, examples of which are given below.

Compositions intended for oral use may be prepared according to any method known to the art for the manufacture of pharmaceutical compositions, and such compositions may contain one or more agents selected from the group consisting of sweetening agents, flavouring agents, colouring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example corn starch or alginic acid; binding agents, for example starch gelatin, acacia, microcrystalline cellulose or polyvinyl pyrrolidone; and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

Aqueous suspensions contain the active materials in admixture with excipients suitable for the manufacture of aqueous suspensions. Such excipients are suspending agents, for example sodium carboxymethylcellulose, methylcellulose, hydroxypropylmethylcellulose, sodium alginate, polyvinyl pyrrolidone, gum tragacanth and gum acacia; dispersing or wetting agents may be a naturally occurring phosphatide, for example lecithin, or condensation products of an alkylene oxide with fatty acids, for example polyoxyethylene stearate, or condensation products of ethylene oxide with long-chain aliphatic alcohols, for example heptadecaethyleneoxycetanol, or condensation products of ethylene oxide with partial esters derived from fatty acids, for example polyoxyethylene sorbitan monooleate. The aqueous suspensions may also contain one or more preservatives, for example ethyl or n-propyl p-hydroxybenzoate, one or more colouring agents, one or more flavouring agents, and one or more sweetening agents, such as sucrose or saccharin.

Oily suspensions may be formulated by suspending the active ingredient in a vegetable oil, for example arachis oil, olive oil, sesame oil or coconut oil, polyoxyethylene hydrogenated castor oil, fatty acids such as oleic acid, or in a mineral oil such as liquid paraffin or in other surfactants or detergents. The oily suspensions may contain a thickening agent, for example beeswax, hard paraffin or cetyl alcohol. Sweetening agents, such as those set forth above, and flavouring agents may be added to provide a palatable oral preparation. These compositions may be preserved by the addition of an antioxidant such as ascorbic acid.

Dispersible powders and granules suitable for preparation of an aqueous suspension by the addition of water provide the active ingredient in admixture with a dispersing or wetting agent, suspending agent and one or more preservatives. Suitable sweetening, flavouring and colouring agents may also be present.

The pharmaceutical compositions of the invention may also be in the form of oil-in-water emulsions. The oily phase may be a vegetable oil, for example olive oil or arachis oil, or a mineral oil, for example liquid paraffin, or mixtures of these. Suitable emulsifying agents may be naturally occurring gums, for example gum acacia or gum tragacanth, naturally occurring phosphatides, for example soya bean, lecithin, and esters or partial esters derived from fatty acids and hexitol anhydrides, for example sorbitan monooleate and condensation products of the said partial esters with ethylene oxide, for example polyoxyethylene sorbitan monooleate. The emulsions may also contain sweetening and flavouring agents.

Syrups and elixirs may be formulated with sweetening agents, for example glycerol, propylene glycol, sorbitol or sucrose. Such formulations may also contain a demulcent, a preservative, flavouring and colouring agents.

Suspensions and emulsions may contain a carrier, for example a natural gum, agar, sodium alginate, pectin, methylcellulose, carboxymethylcellulose, or polyvinyl alcohol.

Any suitable pharmaceutically effective drug which is used for the treatment of a respiratory disease may also be co-administered with compositions of the invention. For example, β₂-agonists, e.g. salbutamol, salmeterol and formoterol, may be formulated for co-administration. Additional anti-muscarinic compounds may also be co-administered. For example, ipratropium (e.g. ipratropium bromide) or tiotropium may be administered.

Additional therapeutic agents, including steroids, may also be co-administered. Examples of suitable steroids include beclomethasone, dipropionate and fluticasone. Other suitable therapeutic agents suitable for co-administration include mucolytics, matrix metalloproteinase inhibitors, leukotrienes, antibiotics, anti-infective agents, antineoplastics, peptides, antitussives, nicotine, PDE4 inhibitors, elastase inhibitors and sodium cromoglycate.

It is particularly preferred that amlexanox should be used in combination with a bronchodilator. Suitable such agents are β-agonists, anti-muscarinics and PDE inhibitors. If used alone, amlexanox may be less suitable for the treatment of allergic asthma.

Amlexanox can be used in combination or co-administration with a wide range of respiratory medicines, with little concern over systemic complications that may arise. Amlexanox can be used in an emergency setting for a condition requiring anti-inflammatory action (e.g. ARDS). The product can be administered on a continued basis without concern over systemic side-effects (e.g. tachycardia).

Amlexanox can be administered on a once daily basis as well as used as a symptom reliever with additional anti-inflammatory activity. The anti-inflammatory activity may be local inflammation in the lung associated with neutrophil reflux.

Depending on the condition to be treated, other agents can be combined with amlexanox. Preferred agents are listed below, for each preferred indication.

### Chronic Rheumatoid Arthritis

Analgesic small molecules; COXI inhibitors, COXII inhibitors, atypical NSAIDs.

Disease-modifying small molecules: Azathioprine, leflunomide, minocycline, corticosteroids, chloroquine, cyclosporine A, hydroxychloroquine, gold salts, penicillamine, methotrexate, sulfasalazine.

Disease-modifying biological therapies: infliximab, etanercept, adalimumab, rituximab, anakinra, tocilizumab, ustekinumab

### Wegener's Granulomatosis

Disease-modifying small molecules; cyclophosphamide, corticosteroids, azathioprine, leflunomide, methotrexate,

Disease-modifying biological therapies: infliximab.

### Behcet's Disease

Disease-modifying small molecules; cyclophosphamide, corticosteroids, azathioprine, chlorambucil, thalidomide.

### Gout Arthritis,

Analgesic small molecules; COXI inhibitors, COXII inhibitors, atypical NSAIDs.

Disease-modifying small molecules; allopurinol and colchicine, sulphinpyrazone.

### Gaucher's Disease

Disease-modifying small molecules; miglustat, isofagomine. Disease-modifying biological therapies; recombinant glucocerebrosidase.

### Cushing's Syndrome

Disease-modifying small molecules; Mifepristone.

### Polycythemia Vera

Disease-modifying small molecules; aspirin, hydroxycarbamide, anagrelide.

Disease-modifying biological therapies; beta-interferon,erlotinib.

### Psoriasis

Disease-modifying small molecules; corticosteroids, Vitamin D analogues, dithranol, tazarotene, methotrexate, acitretin, cyclosporine A, hydroxycarbamide.

Disease-modifying biological therapies; etanercept, adalimumab, infliximab, ustekinumab.

### Inflammatory Bowel Disease

Disease-modifying small molecules; corticosteroids, sulfasalazine, mesalamine, azathioprine, cyclosporine A, metronidazole, ampicillin, sulfonamide, cephalosporin, tetracycline.

Disease-modifying biological therapies; etanercept, adalimumab, infliximab, ustekinumab.

### Thrombosis

Heparin, low molecular weight heparin, warfarin, asparin, ibuprofen.

### Glomerulonephritis

Furosemide, bumetanide, ethacrynic acid, torsemide, enalapril, ramipril, quinapril, perindopril, lisinopril, benazepril, valsartan, telmisartan, losartan, irbesartan, olmesartan, corticosteroids, cyclophosphamide, diazoxide, nitroprusside.

### List of “COXI, COXII and Atypical NSAIDs”

Aceclofenac, acemetacin, alcofenac, alminoprofen, aloxipirin, amfenac, aminophenazone, antraphenine, aspirin, azapropazone, benorilate, benoxaprofen, benzydamine, butibufen, chlorthenoxacine, choline salicylate, chlometacin, dexketoprofen, diclofenac, diflunisal, emorfazone, epirizole, etodolac, feclobuzone, felbinac, fenbufen, fenclofenac, flurbiprofen, glafenine, hydroxylethyl salicylate, ibuprofen, indometacin, indoprofen, ketoprofen, ketorolac, lactyl phenetidin, loxoprofen, mefenamic acid, metamizole, metiazinic acid, mofebutazone, mofezolac, nabumetone, naproxen, nifenazone, niflumic acid, oxametacin, phenacetin, pipebuzone, pranoprofen, propyphenazone, proquazone, protozininc acid, salicylamide, salsalate, sulindac, suprofen, tiaramide, tinoridine, tolfenamic acid and zomepirac.

### List of Corticosteroids

Hydrocortisone, hydrocortisone acetate, cortisone, tixocortol, prednisolone, methylprednisolone, prednisone, triamcinolone acetonide, triamcinolone alcohol, mometasone, amcinonide, budesonide, desonide, fluocinonide, fluocinolone acetonide, halcinonide betamethasone, betamethasone sodium phosphate, dexamethasone, dexamethasone sodium phosphate, fluocortolone hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate, fluprednidene acetate.

The following Examples provides evidence on which the present invention is based.

### EXAMPLE 1

The aim of this Example was to evaluate the effect of orally dosed amlexanox (1-30 mg/kg) compared to dexamethasone (1 mg/ml, i.t.) on LPS-induced neutrophilia in the airways of mice and to determine whether the test compound has an effect at inhibiting airway neutrophilia.

### Study Plan:

### Group Size

n=8
Group 1—sham (control)
Group 2—Vehicle (10% DMSO, p.o.)
Group 3—amlexanox (1 mg/kg, p.o.)
Group 4—amlexanox (3 mg/kg, p.o.)
Group 5—amlexanox (10 mg/kg, p.o.)
Group 6—amlexanox (30 mg/kg, p.o.)
Group 7—dexamethasone (1 mg/ml)
p.o.=per orus (orally)

N.B. All test compounds were administered via oral gavage using a ball tipped stainless steel delivery cannulae or via intra-tracheal dosing (using a PennCentury devise). In all cases this was 1 hour before endotoxin exposure.

### Protocol

Non-fasted mice were weighed, individually identified on the tail with a permanent marker and administered by oral gavage or intra tracheal administration with doses of either vehicle, amlexanox or dexamethasone at time point T=−1 with respect to the start of LPS treatment (see above for group details). At T=0, mice were placed into an exposure chamber and exposed to LPS. The lipopolysaccharide(LPS) was prepared in a solution of 0.5 mg/ml and aerosolised using a De Vibliss ultrasonic nebuliser 2000, so that 7 ml of the solution was aerosolised during the 30 min exposure period.

8 hours after LPS challenge the trachea was cannulated and BALF extracted. The procedure for this involved infusing and withdrawing 1 mL of PBS into the lungs via a tracheal catheter. This procedure was repeated to give a yield of approximately 2 mL lavage fluid. The BALF was aliquoted for analysis of neutrophilia and the remaining BALF stored at −80° C. for future cytokine analysis if required.

Total white cell counts in the BAL fluid samples were measured using a Neubaur haemocytometer.

Cytospin smears of the BAL fluid samples were prepared by centrifugation at 1200 rpm for 2 min at room temperature and stained using a DiffQuik stain system (Dade Behring) from which differential white cells counts were conducted. In all cases the cells were counted blind using oil immersion microscopy.

Data were reported as total and differential number of cells per mL of BALF, mean±S.E.M. (standard error of the mean).

Inter-group deviations were statistically analysed by a one-way analysis of variance (ANOVA). In the case of significant difference in the mean values among the different levels of treatment, comparisons versus the vehicle group were carried out using the Dunnett's test. In case the equal variance test fails, a Kruskal-Wallis one-way analysis of variance on ranks followed by a Dunn's test were used. p<0.05 will be considered statistically significant.

### Compounds and Solutions

Amlexanox dosed via oral gavage was administered in a dose volume of 0.1 ml using a ball tipped stainless steel delivery cannule.

Intra-tracheal dosing of amlexanox and dexamethasone was carried out using a PennCentury FMJ250 microspray delivery device. Both formulations were delivered in

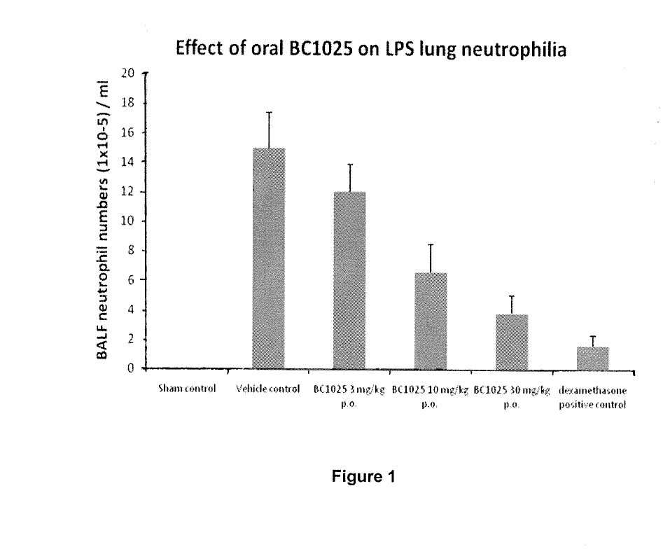

The results are shown in FIG. 1. The results show that amlexanox is effective at inhibiting LPS-induced neutrophilia.

### EXAMPLE 2

The aim of this Example was to evaluate the effect of inhaled amlexanox (0.3-15 mg/mL) and salbutamol (1.0 mg/mL) compared to dexamethasone and fluticasone on LPS-induced neutrophilia in the airways of mice and to determine whether the test compound and salbutamol have a synergistic effect at inhibiting airway neutrophilia.

### Study Plan:

### Group Size

n=8
Group 1—sham
Group 2—vehicle (10% DMSO)
Group 3—amlexanox (0.3 mg/mL)
Group 4—amlexanox (3 mg/mL)
Group 5—amlexanox (10 mg/mL)
Group 6—amlexanox (15 mg/mL)
Group 7—salbutamol (1 mg/mL)
Group 8—amlexanox (0.3 mg/mL)+salbutamol (1 mg/mL)
Group 9—amlexanox (3 mg/mL)+salbutamol (1 mg/mL)
Group 10—dexamethasone (1.0 mg/mL)
Group 11—fluticasone (1.0 mg/mL)
N.B. All test compounds to be administered by intra-tracheal dosing (using a PennCentury device) 1 hour before endotoxin exposure.

### Protocol

Non-fasted mice were weighed, individually identified on the tail with a permanent marker and administered by intra tracheal administration with doses of either vehicle, amlexanox, fluticasone, dexamethasone or salbutamol at time point T=−1 with respect to the start of LPS treatment (see above for group details). At T=0, mice were placed into an exposure chamber and exposed to LPS. The LPS was prepared in a solution of 0.5 mg/ml and aerosolised using a De Vibliss ultrasonic nebuliser 2000, so that 7 ml of the solution was aerosolised during the 30 min exposure period.

8 hours after LPS challenge the trachea was cannulated and BALF extracted. The procedure for this involved infusing and withdrawing 1 mL of PBS into the lungs via a tracheal catheter. This procedure was repeated to give a yield of approximately 2 mL lavage fluid. The BALF was aliquoted for analysis of neutrophilia and the remaining BALF stored at −80° C. for future cytokine analysis if required.

Total and differential white cell counts in the BAL fluid samples were measured using a Neubaur haemocytometer. Cytospin smears of the BAL fluid samples were prepared by centrifugation at 1200 rpm for 1 min at room temperature and stained using a DiffQuik stain system (Dade Behring).

Cells were counted blind using oil immersion microscopy.

Data were reported as total and differential number of cells per mL of BALF, mean±S.E.M. (standard error of the mean).

Inter-group deviations were statistically analysed by a one-way analysis of variance (ANOVA). In the case of significant difference in the mean values among the different levels of treatment, comparisons versus the vehicle group were carried out using the Dunnett's test. In case the equal variance test fails, a Kruskal-Wallis one-way analysis of variance on ranks followed by a Dunn's test were used. p<0.05 will be considered statistically significant.

### Compounds and Solutions

Test compounds were administered in a dose volume of 20 μL using a FMJ-250 PennCentury device for intra-tracheal dosing.

All animals showed a good tolerance of dosing to the vehicle, amlexanox, salbutamol, fluticasone or dexamethasone.

Following LPS aerosol exposure there was a noticeable degree of piloerection in vehicle, amlexanox, salbutamol, fluticasone and dexamethasone treated animals.

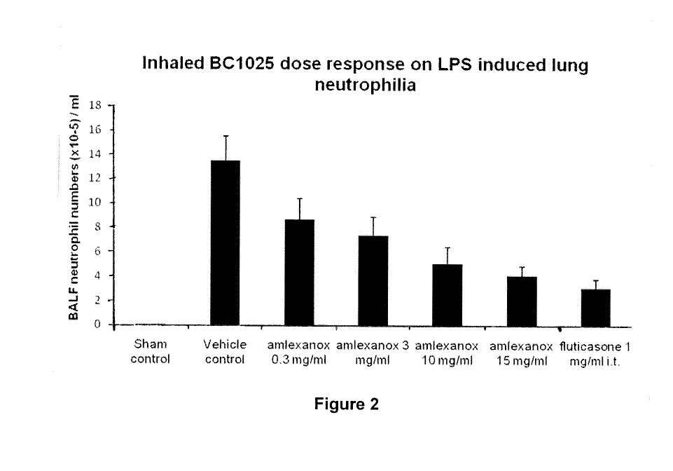

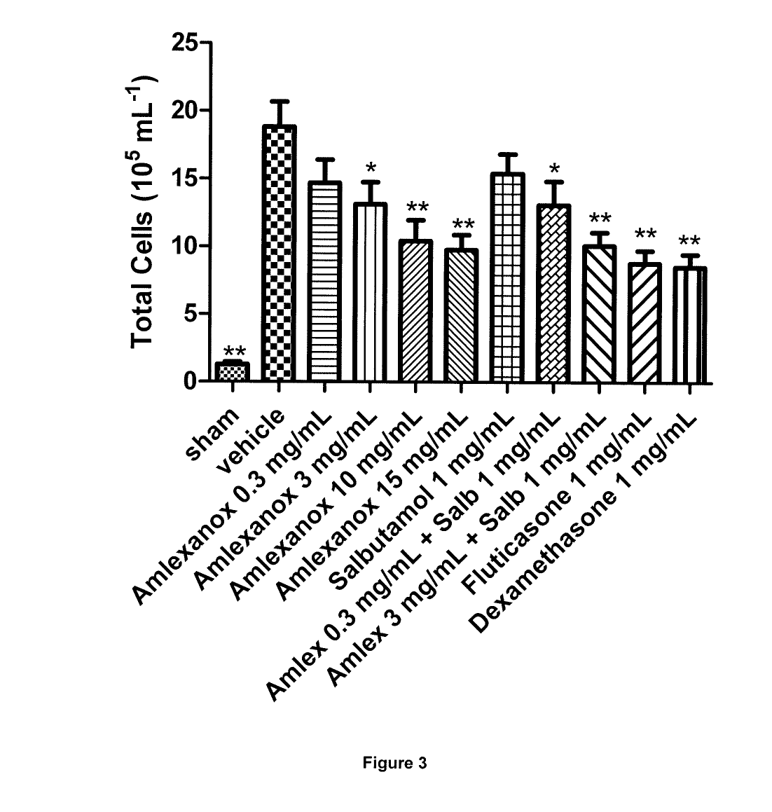

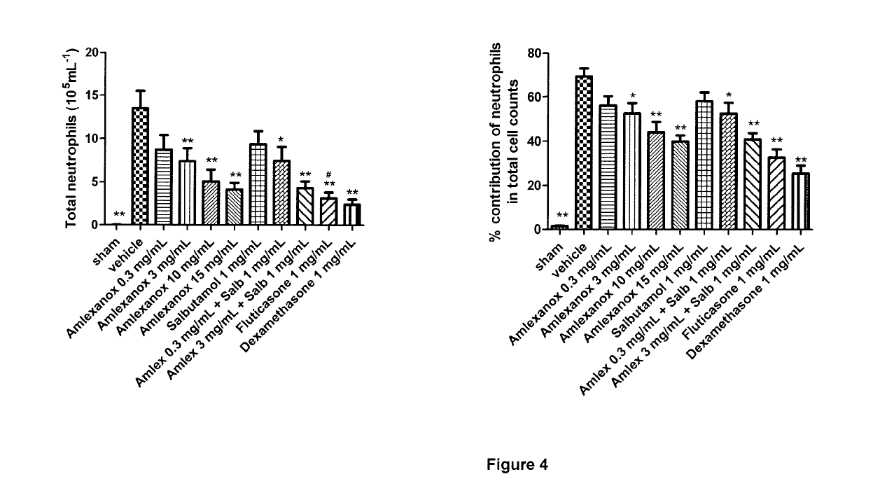

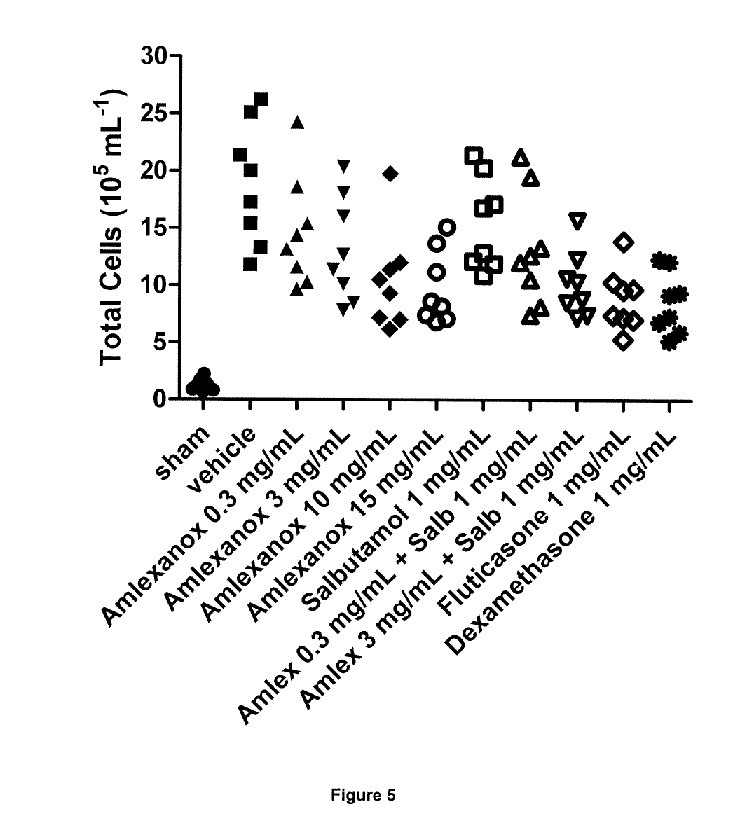

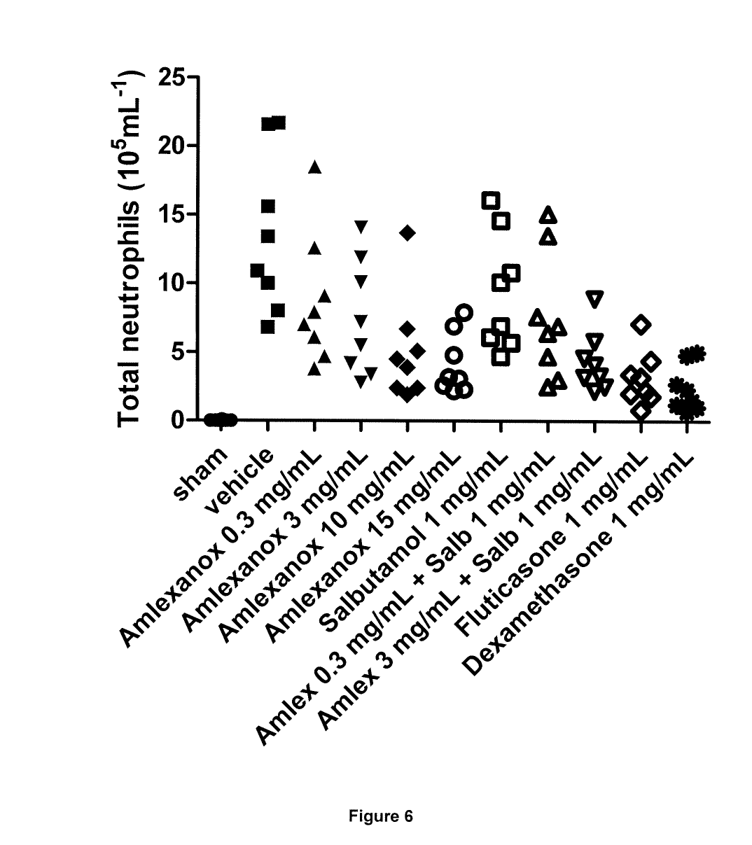

Results are shown in the drawings. FIGS. 2 to 6 show that inhaled amlexanox inhibits neutrophilia.

1. A method for treating a disease associated with neutrophilia, wherein said method comprises administering, to a subject in need of such treatment, amlexanox.

2. The method according to claim 1, wherein the disease is a systemic disease.

3. The method according to claim 2, wherein the disease is an acute infection, a collagen disease, gout, Gaucher's disease, Cushing's syndrome, myelofibrosis, neoplastic neutrophilia, polycythemia vera, psoriasis, inflammatory bowel disease, ischemia reperfusion injury, thrombosis or glomerulonephritis.

4. The method according to claim 3, wherein the ischemia reperfusion injury is a cardiac reperfusion injury, a cerebral reperfusion injury or an organ transplant reperfusion injury.

5. The method according to claim 4, wherein the cardiac reperfusion injury is myocardial infarction.

6. (canceled)

7. The method according to claim 1, wherein the disease is gout.

8. The method according to claim 1 used to treat an irreversible obstructive lung disease.

9. The method according to claim 8, wherein the lung disease is chronic obstructive pulmonary disease (COPD), bronchiectasis, acute respiratory distress syndrome (ARDS), chronic bronchitis, pulmonary emphysema, small airway disease, sarcoidosis or cystic fibrosis.

10. The method according to claim 1, wherein the amlexanox is administered via the oral route or the inhaled route.

11. (canceled)

12. The method according to claim 1, used to treat asthma.

13. A method for treating a respiratory disease wherein said method comprises administering, to a subject in need of such treatment, amlexanox via the inhaled route, provided that the subject of administration is also receiving a bronchodilator if bronchodilation is required for treatment.

14. The method, according to claim 13, used to treat a respiratory disease involving destructive lung inflammation.

15. The method according to claim 13, wherein the disease is asthma and the subject is also receiving a bronchodilator.

16. The method according to claim 13, wherein the respiratory disease is sarcoidosis, COPD, cystic fibrosis ARDS, bronchiectasis, chronic bronchitis, pulmonary emphysema or small airway disease.

17. The method according to claim 13, wherein the respiratory disease is associated with inflammation.

18. The method according to claim 17, wherein the inflammation is indicated by exhaled NO or by the expression of inflammation-related genes (such as IL1b) in peripheral blood neutrophils ex vivo.

19. The method according to claim 13, wherein the respiratory disease is associated with neutrophilia.

20. The method according to claim 13, wherein existing or concomitant treatment with standard anti-inflammatory therapies is judged to provide inadequate benefit or to be leading to undesirable side-effects, including systemic side-effects.

21. (canceled)

22. The method according to claim 1, comprising administering a unit dose in the range from 10 mg to 1 g.

23. (canceled)

24. The method according to claim 13, wherein the amlexanox is in the form of particles having a mass median diameter of up to 10 μm.

25. The method according to claim 13, wherein delivery is achieved using a metered dose inhaler, a dry powder delivery device, or a nebuliser.

26-27. (canceled)

28. A composition comprising particles of amlexanox formulated to be suitable for inhalation as a dry powder, or for inhalation via a pMDI, which is a solution formulation including amlexanox, a propellant, a solvent and water.

29. The composition according to claim 28, which has a fine particle fraction (less than 5 mM) of at least 50%.

30-31. (canceled)

32. The composition according to claim 28, wherein the amlexanox is in the form of particles having a mass median diameter of up to 10 μm.

33. An inhaler device containing a composition of claim 28.

34. A pharmaceutical composition comprising an agent according to claim 28 and a pharmaceutically acceptable carrier, for the treatment of a condition associated with neutrophilia.

35. (canceled)

36. The composition according to claim 35, which does not contain a PPARγ agonist.

37. (canceled)
